(19) United States
(12) Patent Application Publication
(10) Pub. No.: US 2010/0016213 A1
(43) Pub. Date: Jan. 21, 2010

(54) USE OF A DITERPENOID COMPOUND FOR TREATING DIABETES

(75) Inventors: Shoei-Sheng Lee, Taipei (TW); Ming-Jai Su, Taipei (TW)

Correspondence Address: OCCHIUTI ROHLICEK & TSAO, LLP, 10 FAWCETT STREET, CAMBRIDGE, MA, 02138, US

(73) Assignee: National Taiwan University, Taipei (TW)

(21) Appl. No.: 12/504,337

(22) Filed: Jul. 16, 2009

Related U.S. Application Data
(60) Provisional application No. 61/081,183, filed on Jul. 16, 2008.

(51) Int. Cl. A61K 38/28; A61P 3/10; A61K 31/366

(52) U.S. Cl. 514/4; 514/455; 514/453

(57) ABSTRACT

A method for lowering the plasma glucose level in a subject and for treating diabetes with a diterpenoid compound.

### RELATED APPLICATION

This application claims priority to U.S. Provisional Application No. 61/081,183, filed on Jul. 16, 2008, the content of which is hereby incorporated by reference in its entirety.

### BACKGROUND OF THE INVENTION

Herb products derived from Tinospora crispa are widely used for treating various diseases in Asian countries. It has been discovered that furan-type diterpenoids are a major component in these products.

### SUMMARY OF THE INVENTION

This invention is based on the unexpected discovery that two diterpenoid compounds isolated from SDH-V (a species of Tinospora crispa), i.e., borapetoside A and borapetoside C, are effective in treating both type I and type II diabetes.

Accordingly, this invention features a method of treating diabetes by administering to a subject in need of the treatment an effective amount of an isolated compound of formula (I):

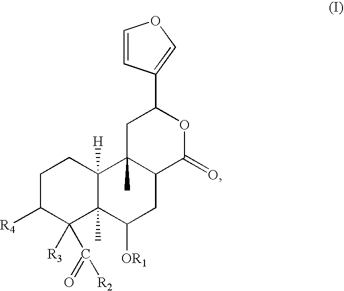

in which R₁ is H or glycosyl, or, together with R₂, forms a bond; R₂ is OH or methoxy, or, together with R₁, forms a bond; R₃ is H or, together with R₄, forms a bond; and R₄ is H or glycosyloxy, or, together with R₃, forms a bond. Optionally, the method of this invention further includes administering to the subject an effective amount of insulin. The term “isolated compound of formula (I)” used herein refers to a compound of formula (I) substantially free from naturally associated molecules, i.e., the naturally associated molecules constituting at most 20% by dry weight of a preparation containing the compound. Purity can be measured by any appropriate method, e.g., HPLC.

In one example, the isolated compound used in the method of this invention is borapetoside A having the formula of:

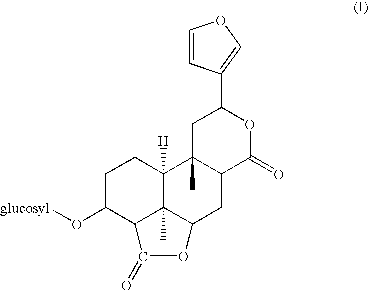

In another example, the isolated compound is borapetoside C having the formula of:

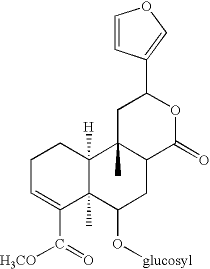

The term “treating” as used herein refers to the application or administration of the compound described herein to a subject, who has diabetes, a symptom of diabetes, or a predisposition toward diabetes, with the purpose to cure, heal, alleviate, relieve, alter, remedy, ameliorate, improve, or affect the disease, the symptoms of the disease, or the predisposition toward the disease. “An effective amount” as used herein refers to the amount of an active agent which, upon administration with one or more other active agents to a subject in need thereof, is required to confer therapeutic effect on the subject. Effective amounts vary, as recognized by those skilled in the art, depending on route of administration, excipient usage, and co-usage with other active agents.

This invention further features a method of lowering the plasma glucose level in a subject who needs this treatment (e.g., a type I or type II diabetic) by administering to the subject an effective amount of the isolated compound described above and optionally insulin.

Also within the scope of this invention is the use of any of the diterpenoid compounds described herein in treating diabetes or in lowering plasma glucose levels, or for the manufacture of a medicament used in the above-mentioned treatments.

The details of one or more embodiments of the invention are set forth in the description below. Other features or advantages of the present invention will be apparent from the following detailed description of several embodiments and also from the appended claims.

### DETAILED DESCRIPTION OF THE INVENTION

Described herein is a method of using a diterpenoid compound of formula (I) for treating diabetes or for lowering the plasma glucose level in a subject (e.g., a human).

The diterpenoid compound of formula I used to practice this invention can be prepared by methods well known in the art, including chemical synthesis and isolation from natural sources.

In one example, the diterpenoid compound is purified from SDH-V by extracting the plant with a suitable solvent (e.g., water, ethanol, or butanol) to obtain an extract and then fractionating the extract via chromatography to obtain the enriched or purified compound. Another example follows. SDH-V is extracted with water to form an aqueous extract, which is then loaded onto a column filled with an adsorbing material (e.g., XAD-II or SP-700). The column is eluted with an alcohol solution (e.g., a methanol or ethanol solution) at a concentration of 70-100% (v/v) to produce a fraction rich in the diterpenoid compound of interest. If necessary, this fraction is further fractionated via, e.g., chromatography, to obtain the diterpenoid compound, in purified form.

A diterpenoid compound of formula I, when obtained from a natural source, can be subsequently modified via chemical reactions to produce other diterpenoid compounds used to practice this invention. As examples, Scheme 1 and Scheme 2 below show synthetic routes of converting borapetoside C and borapetoside A, two naturally-occurring compounds of formula (I), to other diterpenoid compounds of formula (I).

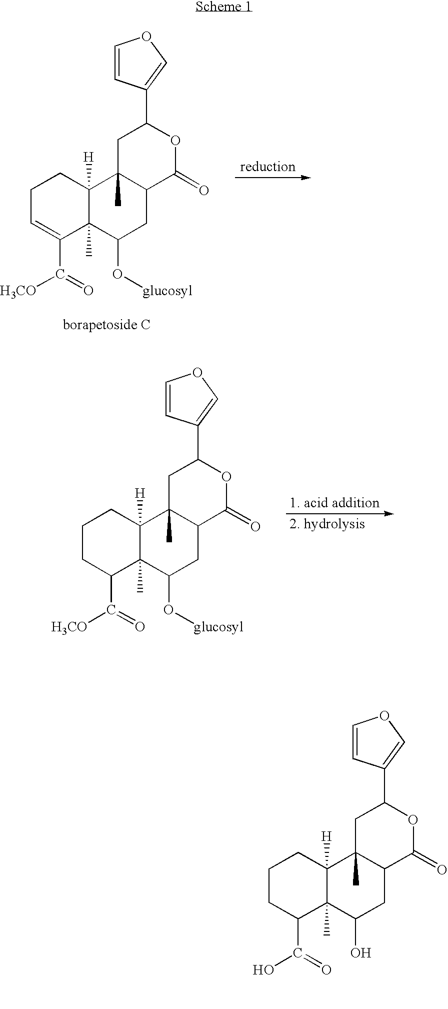

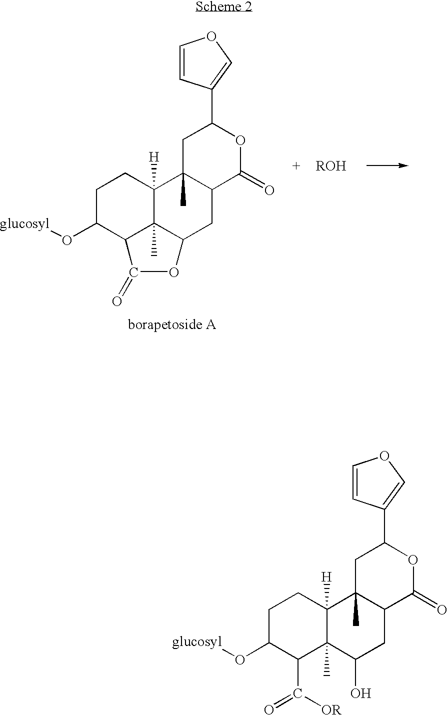

Each of the diterpenoid compounds described herein can be mixed with a pharmaceutically acceptable carrier to form a pharmaceutical composition. A pharmaceutically acceptable carrier is compatible with the active ingredient of the composition, and preferably, capable of stabilizing the active ingredient and not deleterious to the subject to be treated. One or more solubilizing agents can be utilized in the pharmaceutical composition as excipients for delivery the diterpenoid compound. Examples of other carriers include colloidal silicon oxide, magnesium stearate, cellulose, sodium lauryl sulfate, and D&C Yellow #10.

The pharmaceutical composition described above can be used to lower plasma glucose levels or treat diabetes. It can be administered parenterally, enterally (e.g., orally, nasally, and rectally), topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation. A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art.

The pharmaceutical composition described herein can also be administered in the form of suppositories for rectal administration.

The diterpenoid compounds described above can be preliminarily screened for their efficacy in treating diabetes or for lowering plasma glucose levels in an animal model (see Examples 2-8 below) and then confirmed by clinic trials. Other methods will also be apparent to those of ordinary skill in the art.

Without further elaboration, it is believed that one skilled in the art can, based on the above description, utilize the present invention to its fullest extent. The following specific examples are, therefore, to be construed as merely illustrative, and not limitative of the remainder of the disclosure in any way whatsoever. All publications cited herein are incorporated by reference.

### Example 1

### Purification of Borapetoside A and Borapetoside C from SDH-V

50.8 kg stems of SDH-V, a strain of Tinospora crispa, were chopped into small pieces and blended in 30 liter water. After centrifugation, the supernatant was collected and then stored at 5-8° C. for one day to allow formation of precipitates. The precipitates were extracted with ethanol twice, heated at 45° C. for one hour, and then concentrated to obtain an ethanolic extract. The water-soluble fraction was concentrated to form a water extract.

In one approach, 152 g of the ethanolic extract described above were suspended in 1 L water and then extracted sequentially with 0.5 L CH₂Cl₂ three times, 0.5 L ethyl acetate (EA) three times, and 0.5 L n-butanol (n-BuOH) three times, resulting in a CH₂Cl₂-soluble fraction, an EA-soluble fraction, an n-BuOH-soluble fraction, and a water-soluble fraction.

The n-BuOH-soluble fraction was then subjected to centrifugal partition chromatography (CPC), using CHCL₃-MeOH—H₂O (10:10:5) as the stationary phase solvent. The CPC was performed under the following conditions: speed 800 rpm, flow rate 3 mL/min, and temperature 25° C. Eight fractions (Frs 1-8) were obtained from the CPC fractionation. Fr2 was further fractionated by low-pressure column chromatography (Lobar-B, 30-100% MeOH—H₂O) to produce purified borapetoside A, and Fr7 was further fractionated by column chromatography (Sephadex LH-20 column; MeOH—H₂O at 10:3) to produce purified borapetoside C.

In another approach, the water extract (43 g) was loaded onto a column filled with 800 g XAD-II. The column was then eluted sequentially with water, solutions containing methanol at concentrations of 5-10%, 20%, 40%, and 80%, and pure methanol. A borapetoside C-rich fraction was obtained by eluting the column with 100% methanol. Alternatively, the water extract (43 g) was loaded onto a column filled with 200 g SP-700 and borapetoside C was obtained by eluting the column with 100% ethanol.

### Example 2

### Use of Borapetoside A or Borapetoside C for Lowering Plasma Glucose Level in STZ-Induced Diabetic Mice

Normal and streptozotocin (STZ)-induced diabetic mice were administered with borapetoside A (5.0 mg/kg), borapetoside C (5.0 mg/kg), glibenclamide (5.0 mg/kg), metformin (500.0 mg/kg), or a vehicle control via intraperitoneal injection. STZ-induced diabetic mice are a well-known mouse model of type I diabetes (insulin dependent). See, e.g., Liu I M, et al., Neuroscience Letters 2001; 307: 81-84. Glibenclamide and metformin serve as two positive controls in this study.

The plasma glucose levels of the treated mice were determined before and one hour after administration. The activity of lowering plasma glucose levels (A_{L}) is calculated following the formula:

$${A}_{L} = \frac{\begin{matrix}Glucose Level Before Administration - \\ Glucose Level After Administration\end{matrix}}{Glucose Level Before Adminstration} \%$$

Like the two positive controls, both borapetoside A and borapetoside C showed activities in lowering plasma glucose levels in normal and STZ-induced diabetic mice relative to the vehicle control. See Table 1 below. These results are statistically significant (P<0.05).

TABLE 1
Activities of Lowering Plasma Glucose Levels in Normal and STZ-diabetic Mice

| | borapetoside A | borapetoside C | glibenclamide | metformin | vehicle |
|---|---|---|---|---|---|
| normal mice | 49.4 ± 4.8% | 36.2 ± 5.6% | 19.3 ± 1.6% | 20.0 ± 2.5% | 6.0 ± 1.6% |
| STZ-diabetic mice | 54.3 ± 6.3% | 16.5 ± 2.9% | 3.2 ± 1.9% | 34.1 ± 5.5% | 4.4 ± 0.6% |

Borapetoside C was administered to normal mice at 0.1, 1.0, 3.0, and 5.0 mg/kg and to STZ-diabetic mice at 0.1, 1.0, 3.0, and 5.0 mg/kg. The plasma glucose levels of the treated mice were examined before and after treatment. As shown in Table 2 below, borapetoside C lowered plasma glucose levels in both normal and STZ-induced diabetic mice in a dose-dependent manner.

TABLE 2
Plasma Glucose Levels of Normal and STZ-diabetic Mice Treated with
Borapetoside C at Different Doses

| | | Doses of Borapetoside C (mg/kg) | | | | |
|---|---|---|---|---|---|---|
| | | 0.1 | 1.0 | 3.0 | 5.0 | vehicle |
| Normal mice | Before | 99.7 ± 3.8 | 96.4 ± 4.2 | 96.3 ± 8.0 | 98.2 ± 3.9 | 99.3 ± 4.1 |
| (mg/Dl) | After | 97.7 ± 6.2 | 82.6 ± 2.9 | 79.4 ± 6.9 | 67.0 ± 4.5 | 95.8 ± 3.5 |
| STZ-diabetic | Before | 580.0 ± 7.8 | 566.6 ± 11.4 | 572.2 ± 16.2 | 576.0 ± 11.1 | 588.2 ± 9.4 |
| mice (mg/dL) | After | 529.0 ± 14.7 | 472.2 ± 13.9 | 493.8 ± 15.3 | 481.1 ± 20.8 | 567.8 ± 12.1 |

### Example 3

### Use of Borapetoside A or Borapetoside C for Lowering Plasma Glucose Level in Diet-Induced Obesity Diabetic Mice

Normal and diet-induced obesity diabetic mice (“DIO-induced diabetic mice) were administered with borapetoside A (5.0 mg/kg), borapetoside C (5.0 mg/kg), glibenclamide (5.0 mg/kg), metformin (500.0 mg/kg), or a vehicle control via intraperitoneal injection. DIO-induced diabetic mice are a well-known mouse model of type II diabetes (non-insulin dependent). See Liu et al., J Mol. Endocrinol. 2008.

The plasma glucose levels of the treated mice were determined before and one hour after administration. The activities of these compounds for lowering plasma glucose levels were calculated as described above.

The effects borapetoside A and borapetoside C (i.p.) in lowering plasma glucose levels in both normal and DIO-induced diabetic mice were similar to those of glibenclamide and metformin. The A_{L}s of borapetoside A, borapetoside C, glibenclamide, metformin, and vehicle control were 29.7±6.1%, 29.4±1.0%, 25.8±2.1%, 30.7±0.6%, and 6.5±1.1, respectively.

Borapetoside C was administered to DIO-induced diabetic mice at 0.1, 1.0, 3.0, and 5.0 mg/kg and the plasma glucose levels were examined before and after administration. The basal plasma glucose concentration in diet-induced diabetic mice (untreated) was 174.6±7.7 mg/dL. One hour after treatment, the plasma glucose levels in mice treated with vehicle control, 0.1 mg/kg borapetoside C, 1.0 mg/kg borapetoside C, 3.0 mg/kg borapetoside C, and 5.0 mg/kg borapetoside C were 172.5±10.9 mg/dL, 170.5±6.8 mg/dL, 127.9±6.0 mg/dL, 121.3±4.3 mg/dL and 119.9±4.6 mg/dL, respectively. These results indicate that borapetoside C lowered plasma glucose levels in diet-induced diabetic mice in a dose-dependent manner.

### Example 4

### Effect of Borapetoside C for Lowering Plasma Glucose Levels Via Oral Administration

Normal, STZ-induced diabetic mice, and diet-induced diabetic mice were administered orally with 10.0 mg/kg borapetoside C, glibenclamide or metformin as a positive control, and a vehicle control. The plasma glucose levels of the treated mice were determined before and sixty minutes after treatment and the results thus obtained were shown in Table 3 below.

TABLE 3
Activity of Borapetoside C in Lowering Plasma
Glucose Levels Via Oral Administration

| | Activity of lowing plasma |
|---|---|
| Groups | glucose levels (%)^{†} |
| Normal mice | |
| Vehicle | 3.6 ± 3.0 |
| borapetoside C(10.0 mg/kg) | 23.1 ± 2.8** |
| Glibenclamide(10.0 mg/kg) | 25.9 ± 3.5** |
| STZ-diabetic mice (IDDM) | |
| Vehicle-treated | 9.2 ± 1.8 |
| borapetoside C(10.0 mg/kg) | 17.7 ± 1.7** |
| Metformin (500.0 mg/kg) | 18.1 ± 1.9** |
| Diet-induced diabetic mice (NIDDM) | |
| Vehicle | 12.7 ± 4.5 |
| borapetoside C(10.0 mg/kg) | 28.2 ± 3.1* |
| Glibenclamide(10.0 mg/kg) | 34.9 ± 4.0** |

^{†}Values shown in Table 3 are mean ± SEM of A_{L} obtained from six animals in each group.
*p < 0.05
***p < 0.005

### Example 5

### Use of Borapetoside A or Borapetoside C for Stimulating Insulin Release in DIO-Induced Diabetic Mice

Normal mice, STZ-induced diabetic mice, and diet-induced diabetic mice were administered with a vehicle control, borapetoside A (5.0 mg/kg), borapetoside C (at various doses), glibenclamide (5.0 mg/kg) or metformin (500 mg/kg) by intraperitoneal injection. These mice were examined for their blood insulin levels before and one hour after treatment, using the Insulin(Rat) ELISA kit obtained from Penisula Lab. Inc., San Carlos, Calif., USA. The results thus obtained were shown in Table 4 below.

TABLE 4
Plasma Insulin Levels in Mice Before and After Treatment

| | Plasma insulin (pmol/L)^{†} | |
|---|---|---|
| Groups | Pre-treatment | post-treatment |
| Normal mice | | |
| Vehicle-treated | 56.3 ± 3.9 | 61.4 ± 4.9 |
| borapetoside A | 56.0 ± 2.8 | 117.6 ± 10.8** |
| borapetoside C | | |
| 0.1 mg/kg | 54.0 ± 3.4 | 71.5 ± 5.3 |
| 0.5 mg/kg | 52.2 ± 3.0 | 65.7 ± 9.8 |
| 1.0 mg/kg | 58.9 ± 3.5 | 65.0 ± 6.0 |
| 3.0 mg/kg | 52.0 ± 3.7 | 142.6 ± 29.2* |
| 5.0 mg/kg | 56.6 ± 2.3 | 139.4 ± 25.5* |
| Glibenclamide | 50.8 ± 3.6 | 149.5 ± 15.0** |
| STZ-diabetic mice (IDDM) | | |
| Vehicle-treated | 14.4 ± 0.2 | 14.8 ± 0.5 |
| borapetoside A | 16.0 ± 4.1 | 18.7 ± 8.4 |
| borapetoside C | | |
| 0.1 mg/kg | 15.2 ± 0.8 | 16.3 ± 0.9 |
| 0.5 mg/kg | 15.5 ± 0.6 | 15.9 ± 0.6 |
| 1.0 mg/kg | 15.7 ± 0.8 | 15.9 ± 0.4 |
| 3.0 mg/kg | 13.8 ± 1.6 | 14.8 ± 0.5 |
| 5.0 mg/kg | 15.0 ± 0.5 | 14.4 ± 0.6 |
| Metformin | 13.5 ± 1.1 | 15.1 ± 1.5 |
| Diet-induced diabetic (NIDDM) mice | | |
| Vehicle-treated | 117.3 ± 9.1 | 117.3 ± 9.2 |
| borapetoside A | 106.6 ± 14.6 | 173.8 ± 26.9* |
| borapetoside C | | |
| 0.1 mg/kg | 110.3 ± 4.5 | 114.0 ± 7.6 |
| 0.5 mg/kg | 116.5 ± 5.7 | 174.7 ± 4.8** |
| 1.0 mg/kg | 111.1 ± 2.0 | 178.5 ± 14.6** |
| 3.0 mg/kg | 113.2 ± 2.8 | 254.0 ± 23.4** |
| 5.0 mg/kg | 107.4 ± 5.5 | 255.8 ± 15.9** |
| Glibenclamide | 119.3 ± 9.6 | 197.0 ± 18.8** |

^{†}Values shown in Table 4 are mean ± SEM of A_{L} obtained from six animals in each group.
*p < 0.05
**p < 0.005

As shown in Table 4, both borapetoside A and borapetoside C increased plasma insulin levels in normal mice and diet-induced diabetic mice, but not in STZ-induced diabetic mice. In addition, the effect of borapetoside C in improving insulin release was dose dependent.

### Example 6

### Effects of Borapetoside C in Promoting Glycogen Synthesis in Skeletal Muscle and Glucose Tolerance in Diabetic Mice

Normal, STZ-induced diabetic, and DIO-induced diabetic mice were intraeritoneally injected with borapetoside C (5 mg/kg) and Actrapid (0.5 IU/kg; a short-acting insulin provided by Novo Nordisk) or metformin (500 mg/kg). Their glycogen contents in skeletal muscle were determined 30 minutes after injections. As shown in Table 5 below, similar to both metformin and Actrapid, borapetoside C significantly increased glycogen synthesis in both normal and diabetic mice.

TABLE 5
Glycogen Contents in Mice Treated with
Borapetoside C, Insulin, or Metformin

| | Glycogen content |
|---|---|
| Groups | (μmol/g wet weight)^{†} |
| Normal mice | |
| Vehicle-treated | 21.5 ± 0.4 |
| borapetoside C (5 mg/kg) | 25.1 ± 1.3* |
| Insulin (0.5 IU/kg) | 30.2 ± 2.0*** |
| STZ-diabetic mice(IDDM) | |
| Vehicle-treated | 16.7 ± 0.9 |
| borapetoside C (5 mg/kg) | 23.2 ± 0.7*** |
| Insulin (0.5 IU/kg) | 30.0 ± 1.0*** |
| DIO-induced diabetic mice(NIDDM) | |
| Vehicle-treated | 19.2 ± 0.8 |
| borapetoside C (5 mg/kg) | 23.3 ± 0.4*** |
| Insulin (0.5 IU/kg) | 20.2 ± 0.6 |
| Metformin (500 mg/kg) | 23.5 ± 0.9** |

^{†}Values shown in Table 5 are mean ± SEM of A_{L} obtained from six animals in each group.
*p < 0.05
**p < 0.01
***p < 0.005

Normal ICR mice and DIO-induced diabetic mice, treated with borapetoside C (5.0 mg/kg), a vehicle control, or glibenclamide (positive control), were subjected to an intraperitoneal glucose tolerance test (IPGTT) as described in Lamont et al., Diabetes 2008; 57: 190-198. More specifically, glucose (2.0 mg/g body weight) was administered via IP injection to normal and DIO mice and blood samples were drawn from the treated mice before and 30, 60, 120, and 150 min after glucose administration for examination of glucose levels.

In normal mice treated with borapetoside C, the vehicle control, and glibenclamide, the basal plasma glucose concentrations were 106.8±4.7 mg/dL, 114.7±4.6 mg/dL, and 104.9±3.1 mg/dL, respectively. Thirty minutes after intraperitoneal glucose injection, the plasma glucose concentration was elevated to 346.2±14.7 mg/dL in vehicle-treated mice, to 292.9±10.9 mg/dL in borapetoside C-treated mice, or to 273.4±13.6 mg/dL in glibenclamide-treated mice. In other words, 30 minutes after injection of glucose, the plasma glucose level of borapetoside C-treated mice was significantly lower than that of the mice treated with the vehicle. The plasma glucose levels of borapetoside C-treated mice at 60 min, 120 min, and 150 min after treatment were also significantly lower that those of the control mice at the same time points. These results indicate that borapetoside C significantly enhanced in vivo glucose utilization.

In diet-induced diabetic mice treated with borapetoside C, the vehicle control, and glibenclamide, the basal plasma glucose concentrations were 181.6±12.3 mg/dL, 183.1±2.9 mg/dL, and 185.3±7.7 mg/dL, respectively. Thirty minutes after intraperitoneal glucose injection, the plasma glucose concentration was elevated to 405.6±8.9 mg/dL in vehicle-treated mice, to 343.5±11.2 mg/dL in borapetoside C-treated mice, or to 339.1±15.4 mg/dL in glibenclamide-treated mice. Clearly, 30 minutes after injection of glucose, the plasma glucose level of borapetoside C-treated mice was significantly lower than that of the mice treated with the vehicle. The plasma glucose levels of borapetoside C-treated mice at 60 min, 120 min, and 150 min after treatment were also significantly lower that those of the control mice at the same time points. These results indicate that borapetoside C significantly enhanced glucose utilization in non-insulin dependent diabetic mice.

### Example 7

### Effect of Borapetoside C on Insulin Sensitivity in Diabetic Mice

The effect of borapetoside C on insulin sensitivity was tested following the method described in Liu, et al., Clinical and Experimental Pharmacology and Physiology 2005; 32: 649-654. Normal mice (n=6), STZ-induced diabetic mice (n=5), and DIO-induced diabetic mice (n=7) were treated with insulin at various doses (i.e., 0.1 IU/kg, 0.5 IU/kg, and 1.0 IU/kg) together with either borapetoside C (0.1 mg/kg) or a vehicle control. Their plasma glucose levels were examined before and thirty minutes after insulin administration. The activity of lowering plasma glucose levels (A_{L}) was calculated following the formula described in Example 2 above.

TABLE 6
Plasma Glucose Reduction in Diabetic Mice Treated with Insulin and Borapetoside C

| | | A_{L} (%)^{†} | | |
|---|---|---|---|---|
| | | Insulin (0.1 IU/kg) | Insulin (0.5 IU/kg) | Insulin (1.0 IU/kg) |
| Normal mice | Borapetoside C (0.1 mg/kg) | 17.2 ± 1.2 | 45.7 ± 3.7 | 64.7 ± 1.8* |
| | Vehicle | 14.6 ± 0.8 | 37.5 ± 1.1 | 56.8 ± 1.9 |
| STZ-induced | Borapetoside C (0.1 mg/kg) | 10.5 ± 1.4 | 18.9 ± 2.1 | 32.2 ± 2.1** |
| diabetic mice | Vehicle | 8.7 ± 1.6 | 11.5 ± 0.6 | 24.0 ± 1.3 |
| DIO-induced | Borapetoside C (0.1 mg/kg) | 24.0 ± 1.1 | 31.6 ± 1.6 | 42.5 ± 0.8*** |
| diabetic mice | Vehicle | 19.5 ± 1.5 | 27.5 ± 2.6 | 32.0 ± 1.1 |

^{†}Values shown in Table 6 are mean ± SEM of A_{L}
*p < 0.05
**p < 0.01
***p < 0.005

As shown in Table 6 above, insulin reduces the plasma glucose levels in both normal and diabetic mice and the glucose levels were further decreased when the mice were co-administered with borapetoside C. These results indicate that borapetoside C significantly increased sensitivity of diabetic mice to exogenous insulin.

### Example 8

### Effect of Borapetoside C on Liver Glucose Utilization in STZ-Induced Diabetic Mice

To characterize the effect of borapetoside C on liver glucose utilization, levels of liver phosphoenolpyruvate carboxykinase (PEPCK) were examined by western blot analysis in STZ-induced mice treated with borapetoside C (5.0 mg/kg, twice per day for 7 days), insulin, or a vehicle control. Results obtained from this study showed that the PEPCK levels in borapetoside C-treated diabetic mice were similar to those in insulin-treated mice, both much lower that the PEPCK levels in vehicle control-treated mice.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method of treating diabetes, comprising administering to a subject in need thereof an effective amount of an isolated compound of the following formula: 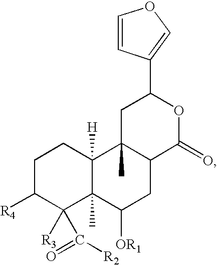
wherein
R₁ is H or glycosyl, or, together with R₂, forms a bond;
R₂ is OH or methoxy, or, together with R₁, forms a bond;
R₃ is H or, together with R₄, forms a bond; and
R₄ is H or glycosyloxy, or, together with R₃, forms a bond.

2. The method of claim 1, wherein the isolated compound is

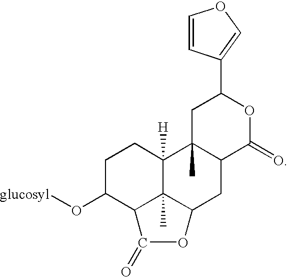

3. The method of claim 1, wherein the isolated compound is

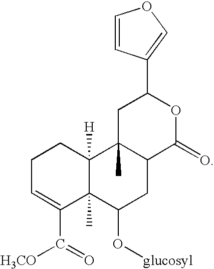

4. The method of claim 1, wherein the subject suffers from type I diabetes.

5. The method of claim 4, wherein the isolated compound is

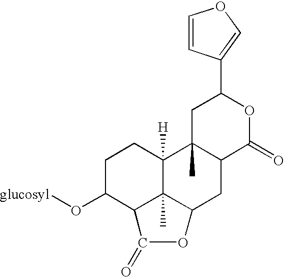

6. The method of claim 4, wherein the isolated compound is

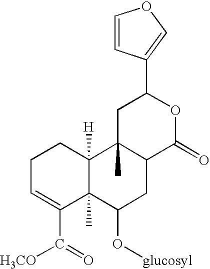

7. The method of claim 1, wherein the subject suffers from type II diabetes.

8. The method of claim 7, wherein the isolated compound is

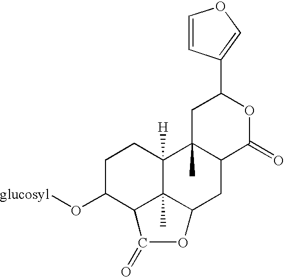

9. The method of claim 7, wherein the isolated compound is

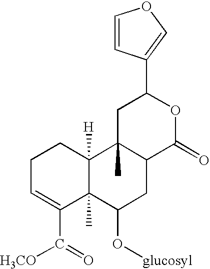

10. The method of claim 1, further comprising administering to the subject an effective amount of insulin.

11. A method of lowering the plasma glucose level in a subject, comprising administering to a subject in need thereof an effective amount of an isolated compound of the following formula: 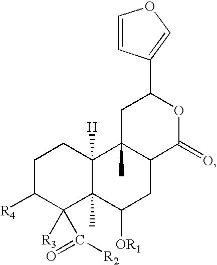
wherein
R₁ is H or glycosyl, or, together with R₂, forms a bond;
R₂ is OH or methoxy, or, together with R₁, forms a bond;
R₃ is H or, together with R₄, forms a bond; and
R₄ is H or glycosyloxy, or, together with R₃, forms a bond.

12. The method of claim 11, wherein the isolated compound is

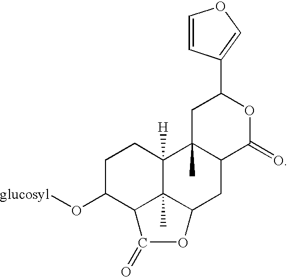

13. The method of claim 11, wherein the isolated compound is

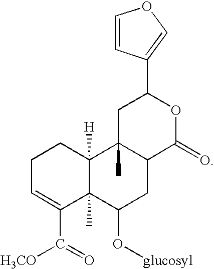

14. The method of claim 11, wherein the subject suffers from type I diabetes.

15. The method of claim 14, wherein the isolated compound is

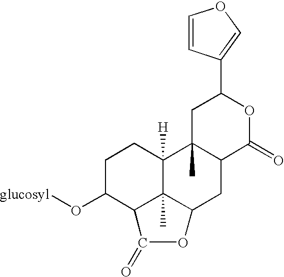

16. The method of claim 14, wherein the isolated compound is

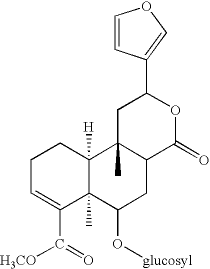

17. The method of claim 11, wherein the subject suffers from type II diabetes.

18. The method of claim 17, wherein the isolated compound is

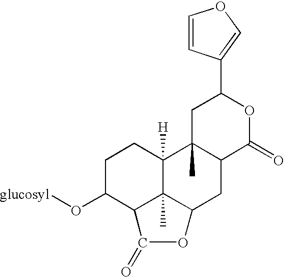

19. The method of claim 17, wherein the isolated compound is

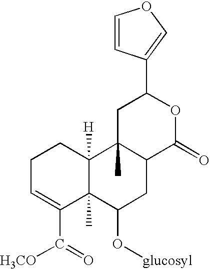

20. The method of claim 11, further comprising administering to the subject an effective amount of insulin.
